(19) United States
(12) Patent Application Publication
(10) Pub. No.: US 2008/0242989 A1
(43) Pub. Date: Oct. 2, 2008

(54) ULTRASONIC IMAGING APPARATUS AND METHOD

(76) Inventor: Tetsuo Koide, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/057,145

(22) Filed: Mar. 27, 2008

(30) Foreign Application Priority Data
Mar. 29, 2007 (JP) 2007-086215

(51) Int. Cl. A61B 8/14; A61B 10/02

(52) U.S. Cl. 600/443; 600/461

(57) ABSTRACT

An ultrasonic imaging apparatus includes: a probe section having a piezoelectric transducer array two-dimensionally arranged in rectangular form on a plane orthogonal to an emitting direction in which an ultrasonic wave is emitted; an image acquisition section which acquires B-mode image information having an imaging cross-section including a scanning direction corresponding to one arrangement direction of the two-dimensional arrangement and the emitting direction, using the probe section; an input unit which inputs an imaging condition for the B-mode image information to the image acquisition section; and a display unit which displays the B-mode image information thereon. The image acquisition section has thickness-direction aperture width switching means which switches an aperture width for performing said emitting in a thickness direction corresponding to another arrangement direction of the two-dimensional arrangement. The input unit has thickness-direction aperture width setting means that sets information about the aperture width to be switched, to the thickness-direction aperture width switching means.

### CROSS REFERENCE TO RELATED APPLICATIONS

This application claims the benefit of Japanese Patent Application No. 2007-086215 filed Mar. 29, 2007, which is hereby incorporated by reference in its entirety.

### BACKGROUND OF THE INVENTION

The subject matter disclosed herein relates to an ultrasonic imaging apparatus that acquires a B-mode image using an ultrasonic probe constituted of a two-dimensionally arranged piezoelectric transducer array and performs biopsy while observing the B-mode image.

An ultrasonic imaging apparatus acquires, in real time, tomographic image information about the position of a subject or subject to be examined, with which an ultrasonic probe makes contact. This real-time property is suitable for confirmation of the position of insertion of a biopsy needle in a subject or subject upon biopsy for inserting the biopsy needle in the subject. The confirmation of the biopsy needle using the ultrasonic imaging apparatus has been widely carried out.

Upon biopsy using the ultrasonic imaging apparatus, a biopsy guide attachment is mounted to the ultrasonic probe, and the biopsy needle is inserted from the end of the ultrasonic probe as viewed in the direction in which electronic scanning is done, along an imaging section. Thus, the position of the biopsy needle from a shallow depth position to a deep depth position is displayed on a B-mode image displayed on the ultrasonic imaging apparatus in the form of a line-shaped emission line.

On the other hand, a plurality of piezoelectric transducers are arranged in the ultrasonic probe having the two-dimensionally arranged piezoelectric transducers even in a thickness direction orthogonal to a scanning direction. Thus, the ultrasonic imaging apparatus controls the number of driven piezoelectric transducers in the thickness direction and a delay time set for every piezoelectric transducer upon transmitting ultrasonic wave, optimizes a focal depth and thickness-direction resolution, and attains an improvement in image quality of the B-mode image (refer to, for example, Japanese Unexamined Patent Publication No. 2000-312676).

According to the background art, however, the biopsy needle to be inserted becomes hard to be displayed on the B-mode image. That is, the biopsy needle to be inserted in the imaging section might be inserted into the position displaced from the imaging section due to allowance of the biopsy guide attachment and flection of the biopsy needle in the subject. The biopsy needle that deviates from the imaging section is not displayed on the B-mode image.

Particularly, when the focal depth is shallow, an aperture width corresponding to the number of driven piezoelectric transducers in the thickness direction is reduced in the ultrasonic probe having the two-dimensionally arranged piezoelectric transducers. In this case, the thickness of the imaging section becomes thin and resolution in the thickness direction is improved at the position near each piezoelectric transducer.

However, the thinning of the imaging section increases the frequency with which the biopsy needle deviates from the imaging section upon its insertion. Further, the number of the piezoelectric transducers in the thickness direction often reaches about 3 to 5 rows in the ultrasonic probe in which the piezoelectric transducers are two-dimensionally arranged. In this case, the thickness of the imaging section is reduced even to about ⅓ to ⅕ by reducing the number of driven piezoelectric transducers. This leads to making it more difficult to insert the biopsy needle into the imaging section.

With these points of view, it is important to determine how to realize an ultrasonic imaging apparatus capable of creating the whole of a biopsy needle lying in a subject in a B-mode image and reliably performing sampling of a subject to be examined by biopsy and its curing or the like even when an ultrasonic probe having a two-dimensionally arranged piezoelectric transducer array is used.

### SUMMARY OF THE INVENTION

It is desirable that the problem described previously is solved.

An ultrasonic imaging apparatus according to the invention of a first aspect includes a probe section having a piezoelectric transducer array two-dimensionally arranged in rectangular form on a plane orthogonal to an emitting direction in which an ultrasonic wave is emitted, an image acquisition section which acquires B-mode image information having an imaging section including a scanning direction corresponding to one arrangement direction of the two-dimensional arrangement and the emitting direction, using the probe section, an input unit which inputs an imaging condition for the B-mode image information to the image acquisition section, and a display unit which displays the B-mode image information thereon, wherein the image acquisition section has thickness-direction aperture width switching means which switches an aperture width for performing the emitting in a thickness direction corresponding to another arrangement direction of the two-dimensional arrangement, and wherein the input unit has thickness-direction aperture width setting means which sets information about the aperture width to be switched, to the thickness-direction aperture width switching means.

In the invention according to the first aspect, the image acquisition section switches the aperture width for emitting the ultrasonic wave in the thickness direction of the two-dimensional arrangement by means of the thickness-direction aperture width switching means. The input unit sets the information about the aperture width to be switched, to the thickness-direction aperture width switching means by means of the thickness-direction aperture width setting means.

An ultrasonic imaging apparatus according to the invention of a second aspect is provided wherein in the ultrasonic imaging apparatus described in the first aspect, the aperture width information includes maximum aperture width information indicative of a maximum aperture width in the thickness direction.

In the invention of the second aspect, the thickness-direction aperture width setting means sets a maximum aperture width and thickens an ultrasonic beam width in the thickness direction.

An ultrasonic imaging apparatus according to the invention of a third aspect is provided wherein in the ultrasonic imaging apparatus described in the first or second aspect, the probe section is equipped with a biopsy guide attachment for inserting a biopsy needle from an end thereof in the scanning direction along the imaging section.

In the invention of the third aspect, biopsy and curing or the like are performed using the ultrasonic imaging apparatus.

An ultrasonic imaging apparatus according to the invention of a fourth aspect is provided wherein in the ultrasonic imaging apparatus described in any one of the first to third aspects, the thickness-direction aperture width setting means designates the aperture width information by the number of piezoelectric transducers for performing the emitting in the thickness direction.

An ultrasonic imaging apparatus according to the invention of a fifth aspect is provided wherein in the ultrasonic imaging apparatus described in any one of the first to fourth aspects, the image acquisition section changes a focal depth position in the thickness direction in sync with the switching of the aperture width.

In the invention of the fifth aspect, the image acquisition section changes a focal depth position in a thickness direction in sync with the switching of an aperture width thereby to optimize the quality of an acquired B-mode image.

An ultrasonic imaging apparatus according to the invention of a sixth aspect is provided wherein in the ultrasonic imaging apparatus described in any one of the first to fifth aspects, the thickness-direction aperture width switching means performs the switching each time one or plural pieces of B-mode image information constituting an image of the imaging section are acquired.

In the invention of the sixth aspect, the switching of the aperture width is not carried out during acquiring one piece of B-mode image information.

An ultrasonic imaging apparatus according to the invention of a seventh aspect is provided wherein in the ultrasonic imaging apparatus described in any one of the first to sixth aspects, the thickness-direction aperture width switching means is provided with thickness-direction aperture width restoring means which performs switching from an aperture width based on initially set aperture width information to an aperture width based on newly set aperture width information simultaneously with the setting and performs re-switching to the initially set aperture width based on the aperture width information after a predetermined time has elapsed from the switching.

In the invention of the seventh aspect, thickening a thickness-direction aperture width for a predetermined time and making it easy to see a biopsy needle by thickening an ultrasonic beam in the thickness direction are assumed temporary.

An ultrasonic imaging apparatus according to the invention of an eighth aspect is provided wherein in the ultrasonic imaging apparatus described in any one of the first to sixth aspects, the thickness-direction aperture width switching means alternately repeatedly switches an aperture width based on initially set aperture width information and an aperture width based on newly set aperture width information simultaneously with the setting.

An ultrasonic imaging apparatus according to the invention of a ninth aspect is provided wherein in the ultrasonic imaging apparatus described in the eighth aspect, the image acquisition section acquires two B-mode images information different in aperture width in the thickness direction in accordance with the switching.

In the invention of the ninth aspect, high-resolution B-mode image information and B-mode image information at which the inserted biopsy needle is clearly seen, are both acquired on a time-sharing basis.

An ultrasonic imaging apparatus according to the invention of a tenth aspect is provided wherein in the ultrasonic imaging apparatus described in the ninth aspect, the display unit simultaneously displays the two B-mode image information.

In the invention of the tenth aspect, two B-mode images different in thickness-direction aperture width are placed in juxtaposed form, and respective beneficial information are read from the two images different in image quality.

According to the invention, when a B-mode image of a biopsy needle is acquired using an ultrasonic probe having a two-dimensionally arranged piezoelectric transducer array, a thickness-direction aperture width through which an ultrasonic wave is emitted, is switched to the maximum aperture width. Therefore, the whole biopsy needle in a subject or body to be examined is reliably created as the B-mode image, by extension, a biopsy subject is reliably detected or sampled from an affected area of each subject or body to be examined.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing an overall configuration of an ultrasonic imaging apparatus.

FIG. 2 is an external view illustrating an external appearance of a probe section.

FIG. 3 is a configurational diagram depicting a configuration of a piezoelectric transducer array built in an ultrasonic probe.

FIG. 4 is a block diagram showing detailed configurations of a piezoelectric transducer array, a B-mode processing unit, a control unit and an input unit.

FIGS. 5(A), 5(B), and 5(C) are explanatory diagrams illustrating a relationship between aperture widths as viewed in thickness directions, focal depth positions and shapes of ultrasonic beams.

FIG. 6 is a flowchart showing operation of an ultrasonic imaging apparatus according to a first embodiment.

FIGS. 7(A) and 7(B) are explanatory diagrams showing a relationship between an aperture width, an entry position of a biopsy needle and a B-mode image as viewed in a thickness direction.

FIGS. 8(A) and 8(B) are explanatory diagrams showing a relationship between an aperture width, an insertion position of a biopsy needle and a B-mode image as viewed in a thickness direction.

FIG. 9 is a block diagram of a control unit according to a second embodiment.

FIG. 10 is an explanatory diagram showing a case in which two B-mode images different in aperture width as viewed in thickness directions are arranged in parallel on a display unit and simultaneously displayed thereon.

### DETAILED DESCRIPTION OF THE INVENTION

Best modes for carrying out an ultrasonic imaging apparatus according to the invention will hereinafter be explained with reference to the accompanying drawings. Incidentally, the invention is not limited thereby.

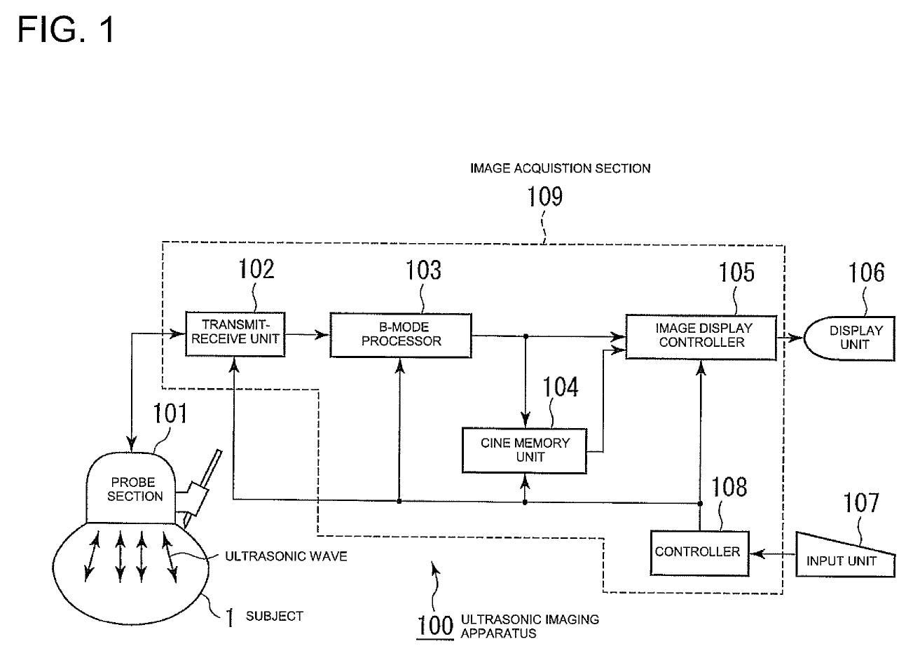

First embodiment. An overall configuration of an ultrasonic imaging apparatus 100 according to a first embodiment will first be explained. FIG. 1 is a block diagram showing the overall configuration of the ultrasonic imaging apparatus 100 according to the first embodiment. The ultrasonic imaging apparatus 100 includes a probe section 101, a transmit-receive unit 102, a B-mode processor 103, a cine memory unit 104, an image display controller 105, a display unit 106, an input unit 107, and a controller 108. Here, the transmit-receive unit 102, the B-mode processor 103, the cine memory unit 104, the image display controller 105 and the control unit 108 constitutes an image acquisition section 109.

The probe section 101 includes an ultrasonic probe for transmitting/receiving an ultrasonic wave, i.e., a portion for emitting the ultrasonic wave to within a subject and receiving each ultrasonic echo reflected from within the subject as a time-series sound ray, and a portion to insert a biopsy needle. Incidentally, the ultrasonic probe includes a piezoelectric transducer array, an acoustic or sound absorbing material, an acoustic or sound matching layer, an acoustic lens, an analog multiplexer, etc. two-dimensionally arranged on a plane as will be described later.

The transmit-receive unit 102 is connected to the probe section 101 by means of a coaxial cable and has pulsers each of which generates a high-voltage electric signal for driving each piezoelectric transducer or element of the probe section 101, and amplifiers each of which initial-stage amplifies the received reflected ultrasonic echo. The transmit-receive unit 102 has a plurality of the pulsers and amplifiers driven with a time difference to carry out electronic focusing.

The B-mode processor 103 is of a part which performs a process for generating a B-mode image from the reflected ultrasonic echo signal amplified by the transmit-receive unit 102 in real time. Specific processing contents include a delay adding process for the received reflected ultrasonic echo signal, an A/D (analog/digital) converting process, a process for writing post-conversion digital information into the image display controller 105 or the cine memory unit 104 to be described later as B-mode image information, etc.

The cine memory unit 104 is of an image memory that stores therein the B-mode image information generated by the B-mode processor 103.

The image display controller 105 performs display frame rate conversion of the B-mode image information generated by the B-mode processor 103 and control on the shape and position of each image display, or the like and outputs the same information to the display unit 106. Here, the image display controller 105 also performs control for simultaneously displaying a plurality of B-mode image information on the display unit 106. For example, the image display controller 105 also outputs the B-mode image information inputted from the B-mode processor 103 to different display regions of the display unit 106 every frame constituting a piece of tomographic image information.

The display unit 106 is constituted of a CRT (Cathode Ray Tube) or an LCD (Liquid Crystal Display) or the like and performs a display of a B-mode image and the like.

The input unit 107 includes a keyboard or a track ball or the like. These constitute scan information input means and thickness-direction aperture width setting means. Scan information, thickness-direction aperture width information and the like are inputted by an operator.

The controller 108 is a part for controlling the operations of the respective parts of the ultrasonic imaging apparatus, based on the scan information and aperture width information inputted from the input unit 107 and programs and data stored in advance.

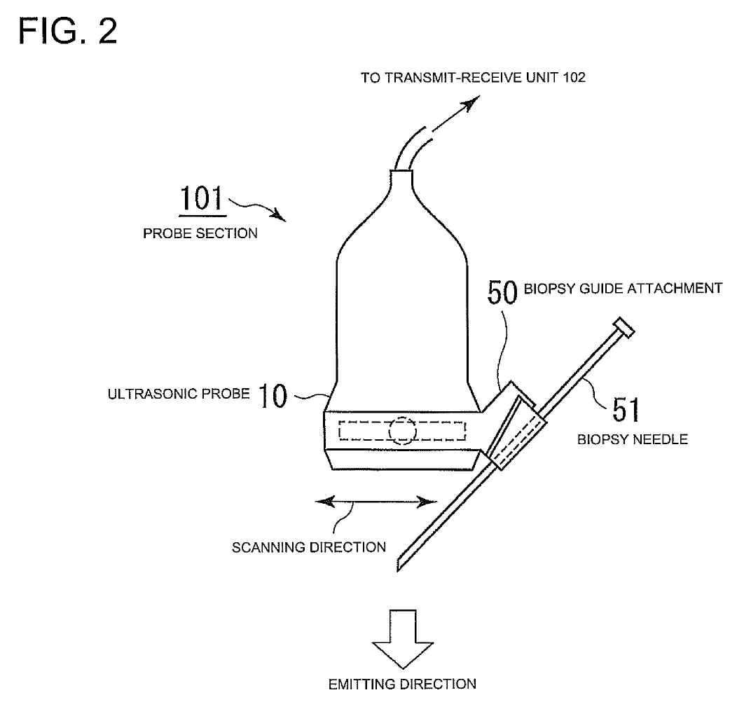

FIG. 2 is an external view showing an outward appearance of the probe section 101. The probe section 101 includes an ultrasonic probe 10, a biopsy guide attachment 50 and a biopsy needle 51. The biopsy guide attachment 50 is mounted onto a holding portion of the ultrasonic probe 10. Incidentally, the biopsy guide attachment 50 is detachably mounted to the holding portion of the ultrasonic probe 10.

The biopsy needle 51 is mounted to the biopsy guide attachment 50. The biopsy needle 51 is mounted to a scan-direction end of the ultrasonic probe 10, for performing its electronic scanning and is positioned at its central portion as viewed in a depth direction thereof orthogonal to the emitting and scanning directions of the ultrasonic probe 10 in such a manner that the biopsy needle 51 is inserted aslant into an imaging section including the emitting and scanning directions of the ultrasonic probe 10.

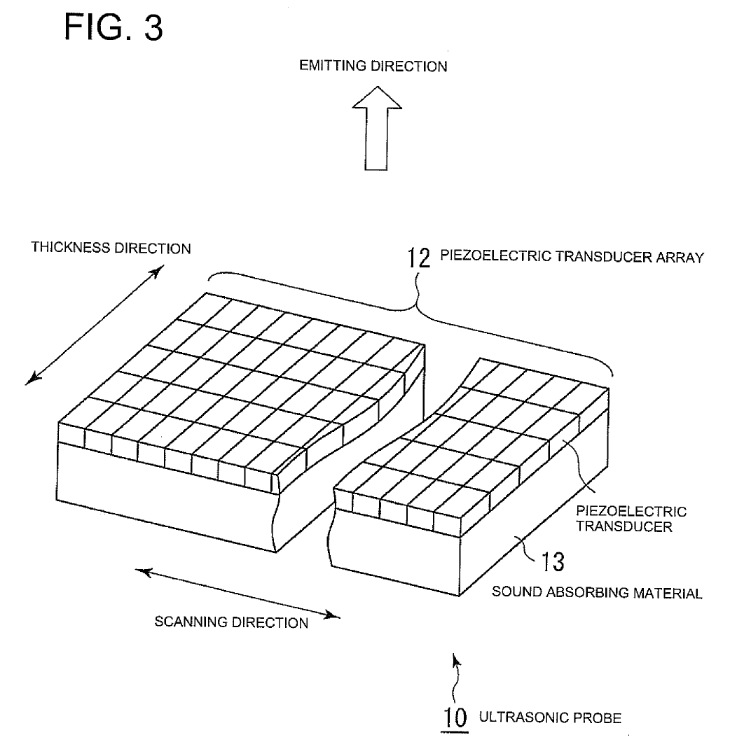

FIG. 3 is a configurational diagram illustrating only the piezoelectric transducer array 12 and acoustic absorbing material 13 contained in the ultrasonic probe 10. A matching layer and a rubber lens unillustrated in the drawing exist in the emitting direction of the piezoelectric transducer array 12. Unillustrated electrodes having shapes pinched or interposed in the emitting direction, and lead electrodes for connecting these electrodes and the analog multiplexer to be described later exist in the piezoelectric transducer array 12 every piezoelectric transducer constituting the piezoelectric transducer array 12.

The piezoelectric transducer array 12 includes a plurality of piezoelectric transducers two-dimensionally arranged in rectangular form on the plane orthogonal to the emitting direction. The piezoelectric transducers are two-dimensionally arranged in the scanning direction in which the electronic scanning is done, and their thickness direction orthogonal to the scanning direction. FIG. 3 shows an example in which piezoelectric transducers corresponding to 5 channels are arranged in their thickness direction and piezoelectric transducers corresponding to about 100 channels are arranged in the scanning direction.

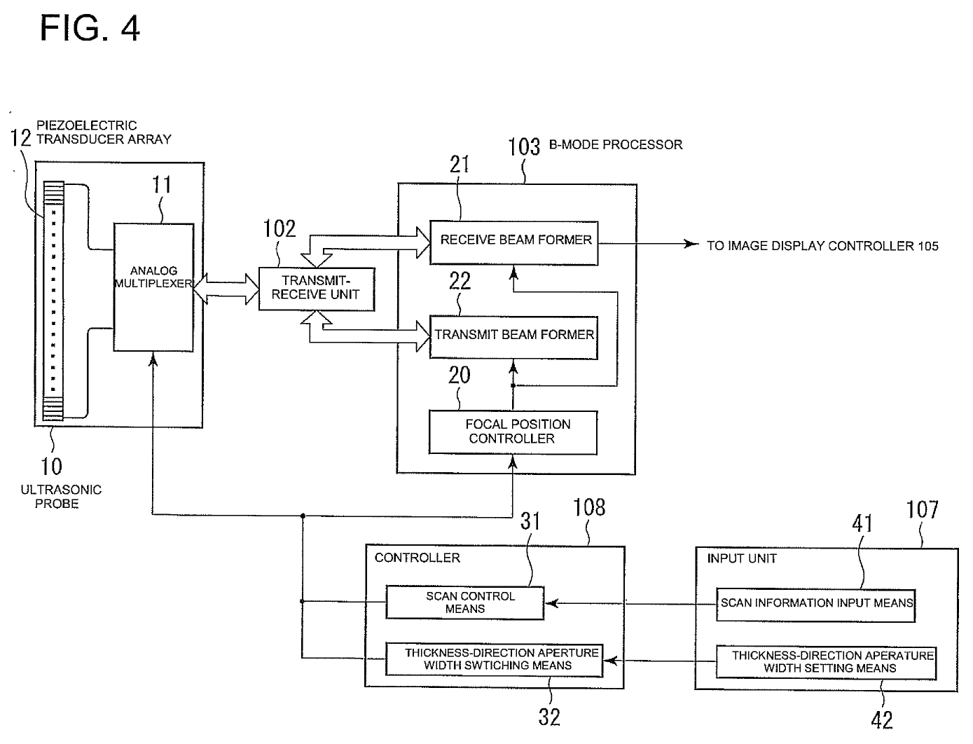

FIG. 4 is a block diagram showing the details of the ultrasonic probe 10, B-mode processor 103, controller 108 and input unit 107, etc. The ultrasonic probe 10 includes the piezoelectric transducer array 12 and the analog multiplexer 11. The B-mode processor 103 includes a receive beam former 21, a transmit beam former 22 and a focal position controller 20. The controller 108 includes scan control means 31 and thickness-direction aperture width switching means 32. The input unit 107 includes scan information input means 41 and thickness-direction aperture width setting means 42.

The transmit beam former 22 forms a trigger signal for driving each pulser of the transmit-receive unit 102. The trigger signal is set in such a manner that an ultrasonic wave produced from each piezoelectric transducer is focused on its corresponding focal depth position of each sound ray as viewed in the emitting direction. Since the ultrasonic probe 10 is of the two-dimensionally arranged piezoelectric transducer array, each focal depth position in the scanning direction and each focal depth position in the thickness direction are set to the transmit beam former 22. The receive beam former 21 dynamically delays and adds reflected ultrasonic echoes received at the piezoelectric transducers are faced in the emitting direction and focused on all points lying on sound rays arranged in the scanning and thickness directions thereby to form a received echo on one sound ray.

In the case of transmission of the ultrasonic wave, the focal position controller 20 calculates the focal depth positions in the scanning and thickness directions and delay times set every piezoelectric transducer for forming the respective sound rays, based on the focal depth positions. When the scan is started, the focal position controller 20 changes delay times using the delay times calculated by the transmit beam former 22 and the receive beam former 21.

The analog multiplexer 11 is of a high withstand analog electronic switch and has input/output terminals one-to-one connected to the piezoelectric transducers of the piezoelectric transducer array 12 and input/output terminals one-to-one connected to the transmit-receive unit 102. The analog multiplexer 11 selectively turns on/off electrical connections between the piezoelectric transducers of the piezoelectric transducer array 12, and the pulsers and amplifiers of the transmit-receive unit 102, based on the scan information and thickness-direction aperture width information sent from the controller 108. With the turning on/off, the piezoelectric transducers connected to their corresponding pulsers are sequentially moved in the scanning direction to perform their scans. Likewise, the number of the piezoelectric transducers in the thickness direction, which are driven by their corresponding pulsers, is also changed according to the turning on/off.

The input unit 107 includes the scan information input means 41 and the thickness-direction aperture width setting means 42. The scan information input means 41 performs the input of scan information, i.e., an imaging range, a focal depth in the scanning direction, etc. using the keyboard or trackball or the like. The thickness-direction aperture width setting means 42 sets the maximum aperture width information in the thickness direction using the keyboard or push buttons or the like. In the ultrasonic probe 10 shown in FIG. 3 by way of example, numerical information of five corresponding to the number of the piezoelectric transducers in the thickness direction can also be inputted as the maximum aperture width information.

The scan control means 31 forms a control signal for performing electronic scanning, based on the scan information transmitted from the input unit 107 and thereby controls the analog multiplexer 11 and the B-mode processor 103. Under their control, the selection of an analog electronic switch for performing electronic scanning is effected on the analog multiplexer 11, whereas the designation of the focal depth positions of the transmit and receive ultrasonic waves are effected on the focal position controller 20.

The thickness-direction aperture width switching means 32 switches the thickness-direction aperture width set to each of the analog multiplexer 11 and B-mode processor 103 as its initial value to the inputted maximum aperture width and a focal depth position suitable for this aperture width, based on the maximum aperture width information in the thickness direction transmitted from the input unit 107.

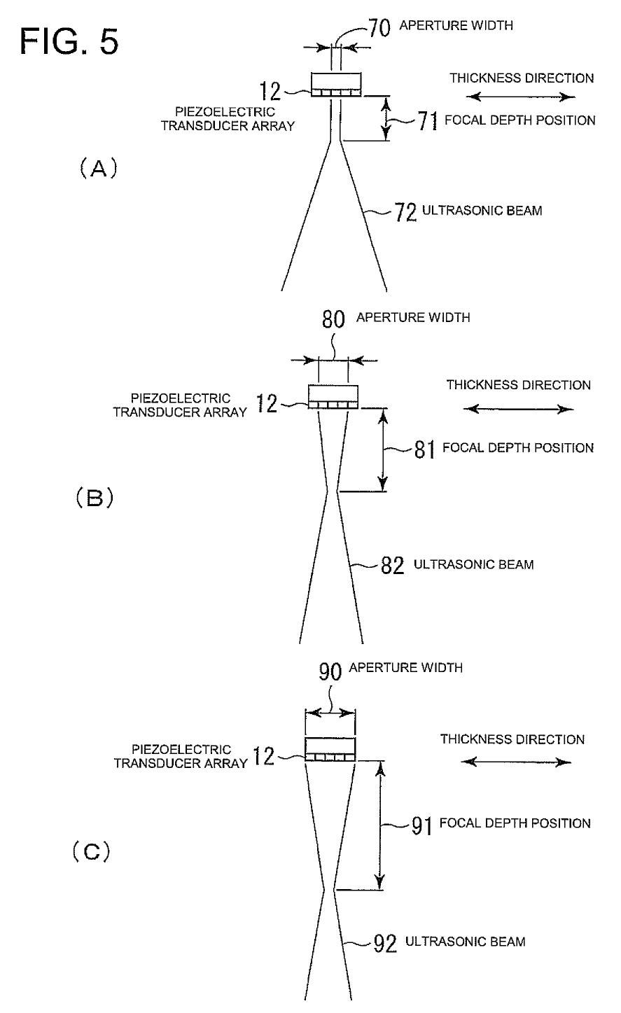

FIGS. 5(A), 5(B), and 5(C) are explanatory diagrams showing thickness-direction aperture widths set as initial values, which are automatically determined in accordance with the focal depth position information set by the operator. The present example illustrates the case of the ultrasonic probe 10 in which the number of piezoelectric transducers in the thickness direction is five similar to FIG. 3. FIGS. 5(A), 5(B), and 5(C) are diagrams each typically showing a thickness-direction section of the piezoelectric transducer array 12 and the shape of an ultrasonic beam emitted from the piezoelectric transducer section. Each of the ultrasonic beams is optimized in such a manner that high image-quality tomographic image information is acquired every focal depth position set by the operator.

FIG. 5(A) illustrates an ultrasonic beam 72 where a focal depth position 71 is placed in a shallow position of a few centimeters. Transmission/reception in the thickness direction is carried out using one piezoelectric transducer positioned at the center. An aperture width 70 as viewed in the thickness direction is assumed to be small. Thus, the thickness in the thickness direction, of the ultrasonic beam 72 is thin up to the focal depth position 71, and a tomographic image having high resolution is acquired. On the other hand, the ultrasonic beam 72 spreads widely at a position deeper than the focal depth position 71, and the resolution in the thickness direction is suddenly degraded.

FIG. 5(B) illustrates an ultrasonic beam 82 where a focal depth position 81 is placed at a middle depth of about 6 to 10 cm. Transmission/reception in the thickness direction is carried out using three piezoelectric transducers positioned in the neighborhood of the center thereof. An aperture width 80 as viewed in the thickness direction is assumed to be middle. Thus, the thickness in the thickness direction, of the ultrasonic beam 82 is gradually narrowed down to the focal depth position 81, and a tomographic image having high resolution is acquired at the focal depth position 81. At a position deeper than the focal depth position 81, the ultrasonic beam 82 spreads gradually with an increase in the depth, and the resolution in the thickness direction is gradually reduced.

FIG. 5(C) illustrates an ultrasonic beam 92 where a focal depth position 91 is placed at a depth of about 10 to 15 cm. Transmission/reception in the thickness direction is carried out using all of five piezoelectric transducers lying in the thickness direction. An aperture width 90 in the thickness direction is assumed to be large. Accordingly, the width in the thickness direction, of the ultrasonic beam 92 becomes wide at a shallow position, and the resolution in the thickness direction is degraded. However, a reduction in resolution is small at a deep focal depth position.

The thickness-direction aperture width switching means 32 switches such initially-set thickness-direction aperture widths 70 and 80 as shown in FIGS. 5(A) and 5(B) to the maximum aperture width 90 shown in FIG. 5(C). The thickness-direction aperture width switching means 32 effects this switching on the analog multiplexer 11 and B-mode processor 103. Upon this switching, the electronic scanning in the scanning direction is not done while one piece of tomographic image information is being acquired. The electronic scanning is done with timing at which the acquisition of the one piece of tomographic image information is terminated. According to this switching, the focal depth positions 71 and 81 are also set to the focal depth position 91 shown in FIG. 5(C).

The thickness-direction aperture width switching means 32 has unillustrated thickness-direction aperture width restoring means. The thickness-direction aperture width restoring means has a timer. When a predetermined time of about a few tens of seconds has elapsed after the switching of the aperture width, the thickness-direction aperture width restoring means resets the switched thickness-direction aperture width to the initial value. Thus, the thickness-direction aperture width restoring means makes thicker the width in the thickness direction, of the ultrasonic beam according to the designation given from the input unit 107 for the predetermined time. Incidentally, with its width thickening, the width in the thickness direction, of the ultrasonic beam becomes thicker for a predetermined time even in the case of a B-mode image displayed on the display unit 106.

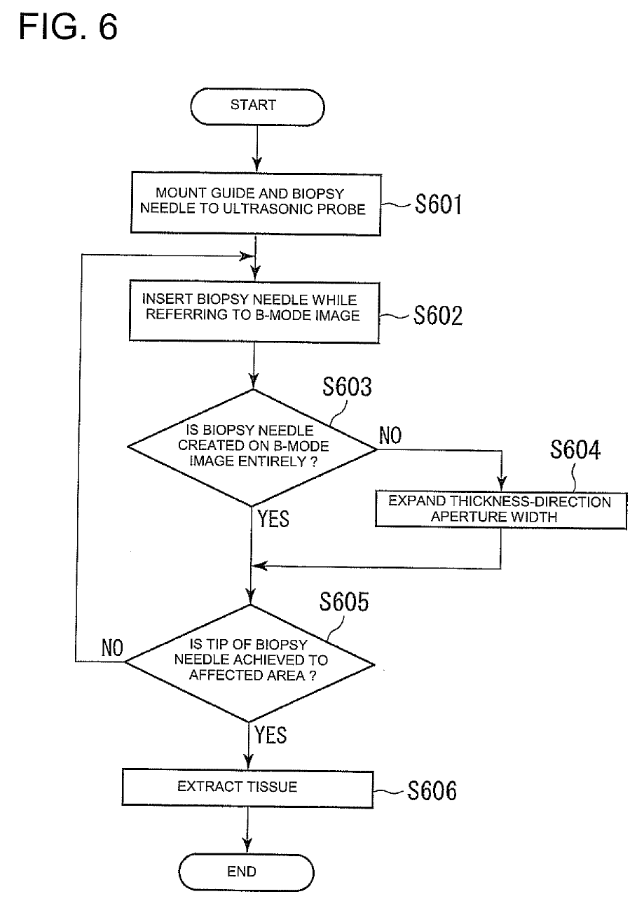

The operation of the ultrasonic imaging apparatus 100 according to the first embodiment will next be explained using FIG. 6. FIG. 6 is a flowchart showing the operation of the ultrasonic imaging apparatus 100 according to the first embodiment.

An operator mounts the biopsy guide attachment 50 and the biopsy needle 51 to the ultrasonic probe 10 (Step S601). Let's now consider a case in which an affected area to be punctured existing inside a subject 1 to be examined is located at a shallow position of about a few centimeters as viewed from the surface of the subject 1, and the operator designates a B-mode image at the shallow focal depth position 71 shown in FIG. 5(A).

Thereafter, the operator inserts the biopsy needle 51 while referring to the B-mode image of the display unit 106 (Step S602). Here, the biopsy guide attachment 50 inserts the biopsy needle 51 from the scanning direction end of the ultrasonic probe 10 along the piezoelectric transducer row positioned in the center in the thickness direction, of the piezoelectric transducer array 12 shown in FIG. 3.

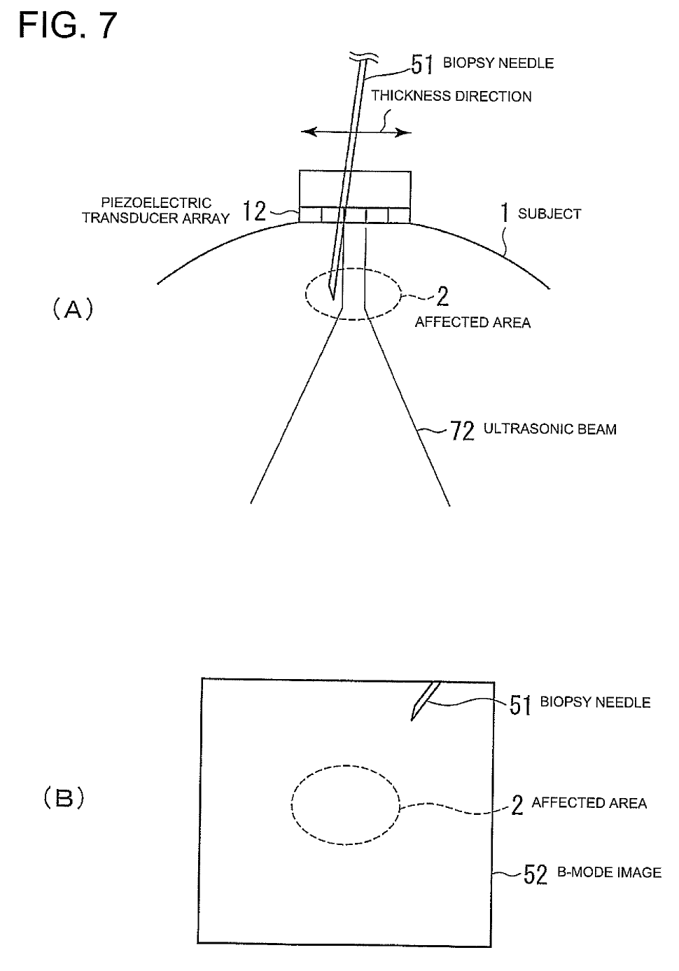

The operator determines whether the biopsy needle 51 is being created on the displayed B-mode image (Step S603). Here, FIGS. 7(A) and 7(B) are explanatory diagrams showing one example of the biopsy needle 51 inserted in the subject 1. FIG. 7(A) is a sectional view of the ultrasonic probe 10 brought close to the subject 1 being in the process of the biopsy needle 51 being inserted therein, as viewed from the thickness-direction section of the piezoelectric transducer array 12 corresponding to the main part. Incidentally, an affected area 2 is placed in a shallow position of about a few centimeters, and the ultrasonic beam at the shallow focal depth position shown in FIG. 5(A) is selected.

FIG. 7(A) illustrates by way of example the case in which the inserted biopsy needle 51 is placed in a position away from a thickness-direction imaging section illustrated in the form of an ultrasonic beam 72. The position to insert the biopsy needle 51 is set to the position placed substantially in the thickness-direction center, by the biopsy guide attachment 50, which position extends along the piezoelectric transducer row. However, the biopsy needle 51 will cause a position displacement from its targeted insertion position due to allowance of the biopsy guide attachment 50 and flection of the biopsy needle 51 itself in the subject 1. Particularly when the affected area 2 is placed in a shallow position of about a few centimeters as viewed from the surface of the subject 1, the biopsy needle 51 is defined as the shallow focal depth position 71. As shown in FIG. 5(A), the aperture width 70 for ultrasonic transmission is small and the ultrasonic wave is generated by only one piezoelectric transducer. Accordingly, the thickness-direction ultrasonic beam 72 that forms the imaging section becomes thin at a shallow depth, and an improvement in resolution is attained. On the other hand, the biopsy needle 51 becomes hard to be inserted into the imaging section.

FIG. 7(B) is an explanatory diagram showing a B-mode image 52 of the display unit 106, which is held in the state of FIG. 7(A). The B-mode image 52 is a tomographic image in a plane orthogonal to the thickness direction, and the image of the affected area 2 is created in its central part. An image of the biopsy needle 51 is partly displayed in the top right of the B-mode image 52. This results from the fact that the biopsy needle 51 shown in FIG. 7(A) is located within the imaging section in the neighborhood of the surface of the subject 1 and is gradually spaced away from the imaging section at a deep position away from the surface.

Referring back to FIG. 6, if the biopsy needle 51 is not created on the displayed B-mode image, the operator thereafter sets the thickness-direction aperture width to the maximum only for a predetermined period using the thickness-direction aperture width setting means 42 of the input unit 107 where the biopsy needle is not fully created in the displayed B-mode image (Step S604).

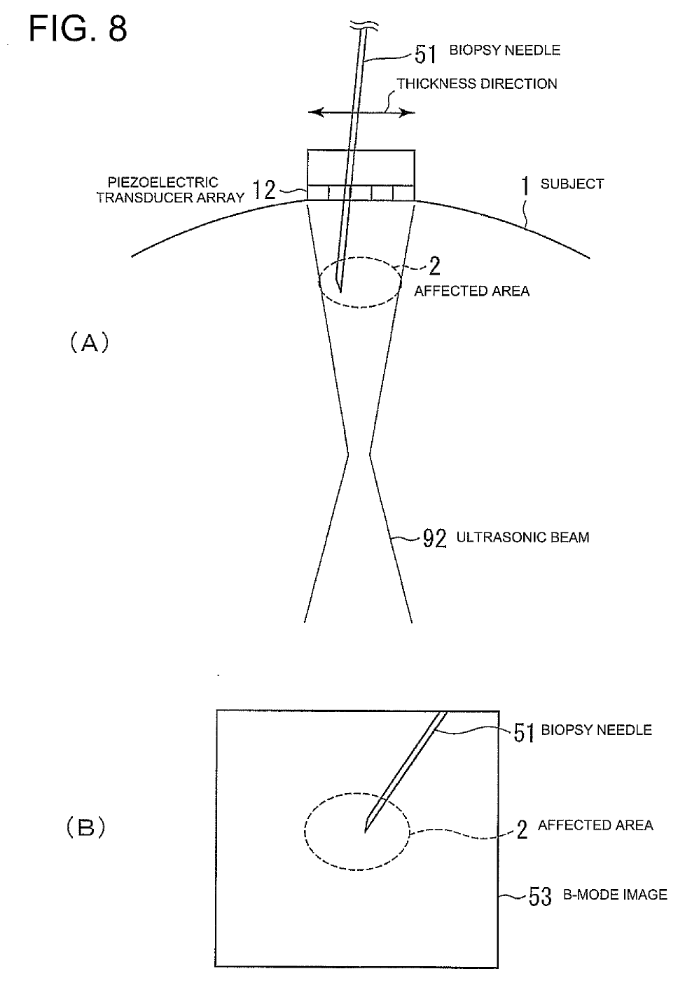

FIGS. 8(A) and 8(B) are explanatory diagrams showing a case in which the thickness-direction aperture width is set maximum under a condition similar to that shown in FIG. 7(A). A thickness-direction aperture width 90 and a focal depth position 91 are similar to those shown in FIG. 5(C). The ultrasonic wave is generated by five piezoelectric elements in the neighborhood of the surface of a subject 1 in which an affected area 2 exists, and the aperture width is set maximum. Accordingly, a thickness-direction ultrasonic beam 92 that forms an imaging section becomes thick at a shallow depth and the resolution is degraded, whereas the biopsy needle 51 can reliably be placed within the imaging section.

FIG. 8(B) shows the manner in which a biopsy needle 51 held in the state of FIG. 8(A) is displayed on a B-mode image 53 of the display unit 106. The B-mode image 53 is of a tomographic image in a plane orthogonal to a thickness direction, and an image of the affected area 2 is displayed in the center thereof. It is understood that an image of the biopsy needle 51 is displayed from the top right of the B-mode image 53 to the affected area 2, and its leading end or tip reaches the affected area 2.

Referring back to FIG. 6, the operator thereafter determines whether the tip of the biopsy needle 51 has reached the affected area 2 (Step S605), because the biopsy needle has been fully created in the displayed B-mode image (Yes in Step S603 and result of Step S604). When the tip of the biopsy needle 51 is found not to have reached the affected area 2 (No in Step S605), the operator proceeds to Step S602, where further insertion of the biopsy needle is done. When the tip of the biopsy needle 51 is found to have reached the affected area 2 (Yes in Step S605), tissue of the affected area is extracted by suction or cutting or the like (Step S606), and the present process is terminated.

In the first embodiment as described above, when the biopsy needle 51 inserted in the subject 1 deviates from the imaging section as viewed in the thickness direction and is not displayed on the B-mode image upon performing the biopsy of the affected area 2 placed in the shallow focal depth position using the two-dimensionally arranged piezoelectric transducer array 12, the aperture width in the thickness direction at the transmission/reception of the ultrasonic wave is made maximum for the predetermined time interval by the thickness-direction aperture width switching means 32 and the thickness-direction aperture width setting means 42 to make the imaging section in the thickness direction thick. Further, the biopsy needle 51 is placed within the imaging section and set so as to be fully displayed in the B-mode image 53. It is therefore possible to reliably grasp the position in the subject 1, of the biopsy needle 51 and perform an error-free biopsy.

In the first embodiment, the thickness-direction aperture width setting means 42 allows the thickness-direction aperture width switching means 32 to set the maximum aperture width. When, however, the initially set aperture width has the minimum aperture width shown in FIG. 5(A), the middle aperture width constituted by the three piezoelectric transducers shown in FIG. 5(B) can also be set to the thickness-direction aperture width switching means 32. In this case, there is a possibility that the width in the thickness direction, of the ultrasonic beam will become thin as compared with the maximum aperture width, and the biopsy needle will deviate from the imaging section. On the other hand, the resolution of the image is improved as compared with the case in which the maximum aperture width is taken, and the affected area 2 becomes easy to see.

Although the first embodiment has illustrated by way of example the case in which the piezoelectric transducers in the thickness direction are five rows, the thickness-direction aperture width can be changed over similarly even where they are constituted of a larger number of piezoelectric transducer rows or sequences. In this case, a plurality of aperture widths broader than the initially set aperture width can be set as the aperture widths set by the thickness-direction aperture width setting means 42. The aperture width is not necessarily limited to the maximum aperture width.

Second embodiment. On the other hand, although in the first embodiment, the thickness-direction aperture width is set maximum for the predetermined time to locate the biopsy needle 51 within the imaging section in the thickness direction and confirm the position of the biopsy needle 51 on the B-mode image, the initial value of the thickness-direction aperture width and the maximum value thereof are alternately switched each time one piece of B-mode image information is acquired, to obtain two B-mode images different in aperture width, whereby they can also be displayed simultaneously. Thus, the second embodiment shows the case in which two B-mode images different in initial or maximum value in terms of a thickness-direction aperture width are acquired.

Here, an ultrasonic imaging apparatus according to the second embodiment is exactly the same as the ultrasonic imaging apparatus described in FIGS. 1 through 4 except for the controller 108 and other explanations will therefore be omitted.

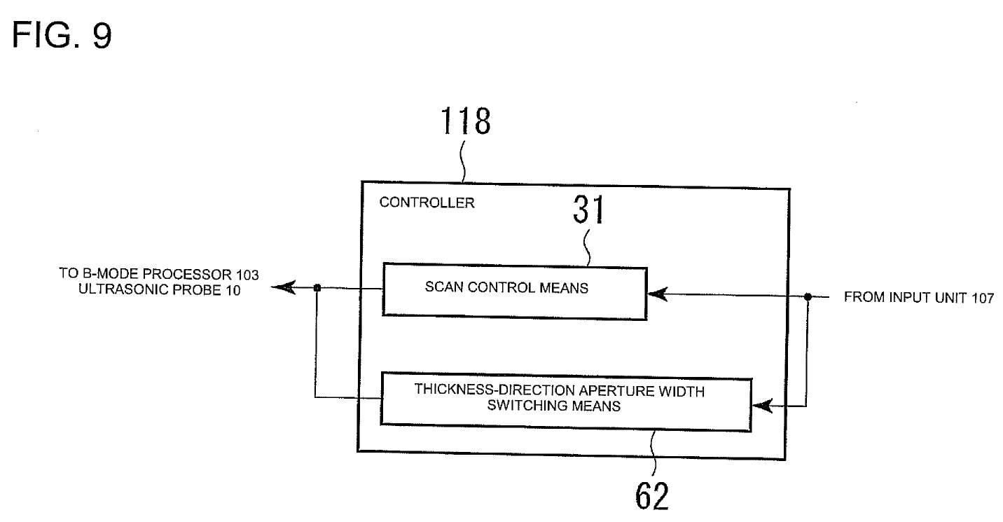

FIG. 9 is a block diagram of a controller 118 according to the second embodiment. The controller 118 includes scan control means 31 and thickness-direction aperture width switching means 62. Since the scan control means 31 is exactly the same as one described in the first embodiment, its explanations are omitted.

The thickness-direction aperture width switching means 62 alternately sets initial value aperture width information and maximum aperture width information to an analog multiplexer 11 and a B-mode processor 103, based on the maximum aperture width information sent from thickness-direction aperture width setting means 42, each time one piece of B-mode image information is acquired, thereby to acquire a B-mode image.

An image display controller 105 controls two B-mode image information different in thickness-direction aperture width distinctively for each frame corresponding to the one piece of B-mode image information and displays the two B-mode image information different in aperture width on a display unit 106 in a juxtaposed state.

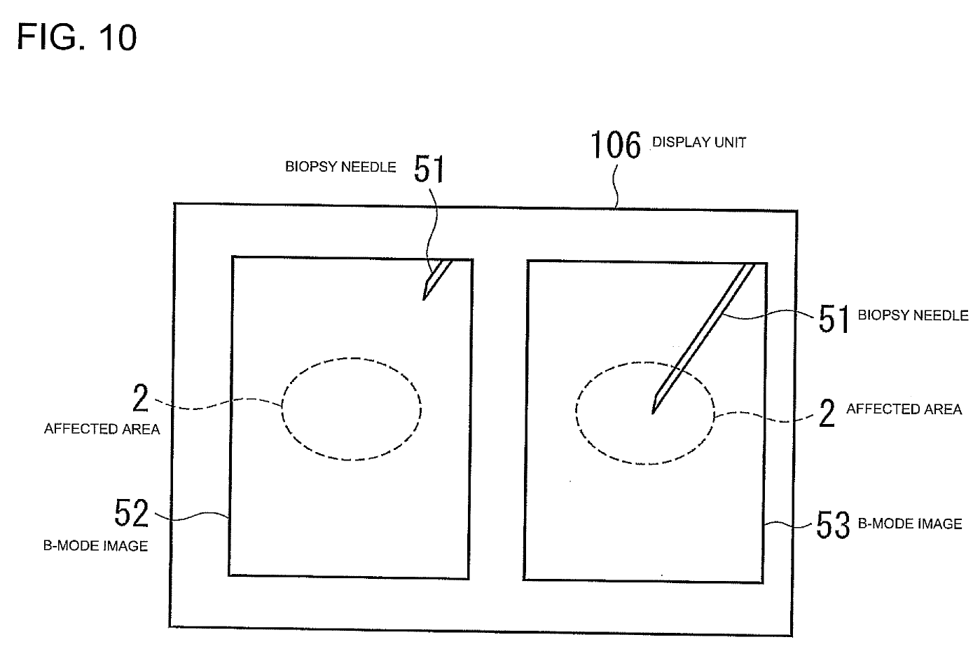

FIG. 10 shows an example illustrative of two B-mode images displayed on the display unit 106, based on imaging conditions similar to those shown in FIGS. 7(A), 7(B), 8(A), and 8(B). A B-mode image 52 in which a thickness-direction aperture width is an initial value, is displayed on the left side of the screen of the display unit 106, whereas a B-mode image 53 in which a thickness-direction aperture width is of a maximum value is displayed on the right side of the screen of the display unit 106.

Here, since the B-mode image 52 is narrow in thickness-direction aperture width and high in resolution, an image of an affected area 2 is clearly created, whereas an image of a biopsy needle 51 is partly created. Since the B-mode image 53 is thick in thickness-direction aperture width and low in resolution, an image of an affected area 2 is unclearly created, whereas an image of a biopsy needle 51 is fully created up to its leading end or tip.

In the second embodiment as described above, the two B-mode images different in thickness-direction aperture width are displayed on the display unit 106 in juxtaposed form. It is therefore possible to simultaneously observe the image in which the affected area 2 has been created in high resolution and the image in which the biopsy needle 51 has been fully created up to its tip, and to insert the error-free biopsy needle 51 into the affected area 2.

Many widely different embodiments of the invention may be configured without departing from the spirit and the scope of the present invention. It should be understood that the present invention is not limited to the specific embodiments described in the specification, except as defined in the appended claims.

1. An ultrasonic imaging apparatus comprising:
a probe section comprising a piezoelectric transducer array two-dimensionally arranged in rectangular form on a plane orthogonal to an emitting direction in which an ultrasonic wave is emitted;
an image acquisition section configured to acquire B-mode image information having an imaging cross-section including a scanning direction corresponding to one arrangement direction of the two-dimensional arrangement and the emitting direction, using said probe section;
an input unit configured to input an imaging condition for the B-mode image information to the image acquisition section; and
a display unit configured to display the B-mode image information,
wherein said image acquisition section comprises a thickness-direction aperture width switching means configured to switch an aperture width for emitting the ultrasonic wave in a thickness direction corresponding to another arrangement direction of the two-dimensional arrangement, and
wherein said input unit comprises a thickness-direction aperture width setting means configured to communicate information about the aperture width to be, switched to said thickness-direction aperture width switching means.

2. The ultrasonic imaging apparatus according to claim 1, wherein the aperture width information includes maximum aperture width information indicative of a maximum aperture width in the thickness direction.

3. The ultrasonic imaging apparatus according to claim 1, wherein said probe section further comprises a biopsy guide attachment configured to insert a biopsy needle from an end of said body guide attachment in the scanning direction along said imaging section.

4. The ultrasonic imaging apparatus according to claim 2, wherein said probe section further comprises a biopsy guide attachment configured to insert a biopsy needle from an end of said body guide attachment in the scanning direction along said imaging section.

5. The ultrasonic imaging apparatus according to claim 1, wherein said thickness-direction aperture width setting means is configured to designate the aperture width information by a number of piezoelectric transducers of said piezoelectric transducer array for emitting the ultrasonic wave in the thickness direction.

6. The ultrasonic imaging apparatus according to claim 2, wherein said thickness-direction aperture width setting means is configured to designate the aperture width information by a number of piezoelectric transducers of said Piezoelectric transducer array for emitting the ultrasonic wave in the thickness direction.

7. The ultrasonic imaging apparatus according to claim 3, wherein said thickness-direction aperture width setting means is configured to designate the aperture width information by a number of piezoelectric transducers of said piezoelectric transducer array for emitting the ultrasonic wave in the thickness direction.

8. The ultrasonic imaging apparatus according to claim 1, wherein said image acquisition section is configured to change a focal depth position in the thickness direction in sync with the switching of the aperture width.

9. The ultrasonic imaging apparatus according to claim 2, wherein said image acquisition section is configured to change a focal depth position in the thickness direction in sync with the switching of the aperture width.

10. The ultrasonic imaging apparatus according to claim 3, wherein said image acquisition section is configured to change a focal depth position in the thickness direction in sync with the switching of the aperture width.

11. The ultrasonic imaging apparatus according to claim 5, wherein said image acquisition section is configured to change a focal depth position in the thickness direction in sync with the switching of the aperture width.

12. The ultrasonic imaging apparatus according to claim 1, wherein said thickness-direction aperture width switching means is configured to switch the aperture width each time at least one piece of B-mode image information constituting an image of the imaging section is acquired.

13. The ultrasonic imaging apparatus according to claim 2, wherein said thickness-direction aperture width switching means is configured to switch the aperture width each time at least one piece of B-mode image information constituting an image of the imaging section is acquired.

14. The ultrasonic imaging apparatus according to claim 3, wherein said thickness-direction aperture width switching means is configured to switch the aperture width each time at least one piece of B-mode image information constituting an image of the imaging section is acquired.

15. The ultrasonic imaging apparatus according to claim 5, wherein said thickness-direction aperture width switching means is configured to switch the aperture width each time at least one piece of B-mode image information constituting an image of the imaging section is acquired.

16. The ultrasonic imaging apparatus according to claim 1, wherein said thickness-direction aperture width switching means comprises a thickness-direction aperture width restoring means configured to switch from a first aperture width based on initially set aperture width information and a second aperture width based on newly set aperture width information simultaneously with the setting of the newly set aperture width and further configured to switch from the second aperture width to the first aperture width after a predetermined time has elapsed after switching from the first aperture width to the second aperture width.

17. The ultrasonic imaging apparatus according to claim 1, wherein said thickness-direction aperture width switching means is configured to alternately and repeatedly switch between a first aperture width based on initially set aperture width information and a second aperture width based on newly set aperture width information simultaneously with the setting of the newly set aperture width.

18. The ultrasonic imaging apparatus according to claim 17, wherein said image acquisition section is configured to acquire two B-mode images information different in aperture width in the thickness direction in accordance with switching between the first aperture width and the second aperture width.

19. The ultrasonic imaging apparatus according to claim 18, wherein said display unit is configured to simultaneously display the two pieces of B-mode image information.

20. A method for ultrasonic imaging, comprising:
transmitting a ultrasonic beam to a subject and receiving ultrasonic waves, by using a probe;
creating a B-mode image;
inserting a biopsy needle into the subject; and
expanding a thickness-direction aperture width for transmitting the ultrasonic beam.
